(19) United States
(12) Patent Application Publication
(10) Pub. No.: US 2021/0355100 A1
(43) Pub. Date: Nov. 18, 2021

(54) Glycolide Production With Low Solid Residue

(71) Applicant: Pujing Chemical Industry Co., Ltd., Shanghai (CN)

(72) Inventor: Wei LIU, Shanghai (CN)

(21) Appl. No.: 17/289,430

(22) PCT Filed: Oct. 29, 2018
(86) PCT No.: PCT/CN2018/112473

§ 371 Date: Apr. 28, 2021

(51) Int. Cl. C07D 319/12

(52) U.S. Cl. CPC C07D 319/12

(57) ABSTRACT

The invention relates a process of preparing glycolide from a methyl glycolate oligomer. The process comprises pyrolyzing a methyl glycolate oligomer in a pyrolysis reaction system. The pyrolysis reaction system comprises no more than 1 wt % of a polyester, a polyol, a polyacid or a combination thereof, based on the total weight of the methyl glycolate oligomer in the system. Also provided is a composition comprising greater than 90 wt % of methyl glycolate and no more than 1 wt % of a combination of the polyester, the polyol, and the polyacid, based on the weight of the composition.

### FIELD OF THE INVENTION

The present invention relates to a process of preparing glycolide production from a methyl glycolate oligomer.

### BACKGROUND OF THE INVENTION

Methyl polyglycolate has good biocompatibility, excellent gas barrier properties, excellent mechanical properties, and excellent biodegradability. It is a polymer material with great potential and green environmental protection, which is in line with the development of today's society. The direction of demand is towards wide used in biomedical devices, shale gas mining, packaging materials and other fields. Because methyl polyglycolate having commercial market value is prepared by ring-opening polymerization of glycolide, the price and purity of the resulting glycolide determine directly the price and quality of the methyl polyglycolate products. Currently all industrial glycolide production is accomplished by prepolymerization, pyrolysis and purification of glycolic acid esters, or glycolic acid or glycolate. The entire glycolide production process is long, especially the pyrolysis process. The efficiency and product quality from this step directly determine the cost of the entire production process.

A few methods for preparing glycolide by depolymerization of a glycolide oligomer have been proposed so far. For example, U.S. Pat. No. 2,668,162A reports a process for preparing high molecular weight polyhydroxycarboxylic acids by breaking a glycolic acid oligomer into a powder under ultra-high vacuum (12-15 Torr (1.6-2.0 kPa)). It was heated at 270-285° C. and slowly fed to the reaction vessel (about 20 g/hour) to depolymerize, and the resulting vapor containing glycolide was collected. Although this method is suitable for small-scale production of glycolide, it is difficult to achieve mass production and is therefore not suitable for industrial production. Further, the method causes the oligomer to become heavy when heated, so that a large amount of the raw materials and the residue remain as a solid residue in the reaction vessel, resulting in a decrease in the yield of glycolide and it's necessary to periodically remove the residue. U.S. Pat. No. 5,326,887 provides a method for preparing glycolide in a catalyst bed, but the reaction apparatus is not easy to clean and the yield is low. U.S. Pat. Nos. 5,091,544, 5,117,008, 5,266,706 report the introduction of an inert gas stream to increase the reaction interface so that promote the rapid conversion of oligomers to cyclic monomers and recovery of cyclic monomers from a gas stream using a water-insoluble, non-polar solvent. However, because the rate of formation of cyclic dimers is slower, this method has low production efficiency and it is difficult to reduce production costs and achieve industrial production.

A process for preparing glycolide by azeotropic method is described in CN104163809A. The para- or meta-aromatic dicarboxylic acid diester is mixed as an azeotropic solution with a glycolic acid oligomer, a catalyst and a co-solvent, and are heated under normal pressure or reduced pressure to cause the glycolic acid oligomer to occur. After depolymerization, co-distillation of glycolide and azeotropic solvent, glycolide obtained from the distillate, and recovery of azeotropic solvent, the yield of glycolide can reach 85%. The azeotrope method involves the distillation and recovery of a large amount of azeotropic solvent, resulting in great waste. At the same time, the addition of a high-boiling solvent also places high demands on the preparation temperature of glycolide. When the temperature is too high, the organic solvent can easily form waste solid residues, which also increase the production cost of glycolide. Accordingly to these patents, it is difficult to increase the yield of glycolide to more than 90%. The low yield means not only low production efficiency and unguaranteed product quality, but also means that additional solid waste will cause blockage of pipelines and create maintenance issues. The downtime caused by the cleaning of waste solid residues will directly lead to the investment of time and labor costs. At the same time, the treatment of solid waste will cause certain environmental pressures, which is not in line with the development trend of today's society. In addition, most patents do not indicate the cause of the solid residue production.

There remains a need for highly efficient processes for producing low-cost, low-solid residue, and high-purity glycolide on an industrial scale.

### SUMMARY OF THE INVENTION

The present invention relates to a process for producing glycolide from a methyl glycolate oligomer and a composition comprising methyl glycolate.

A process of preparing glycolide from a methyl glycolate oligomer is provided. The process comprises pyrolyzing a methyl glycolate oligomer in a pyrolysis reaction system, wherein the pyrolysis reaction system comprises no more than 1 wt % of a polyester, a polyol, a polyacid or a combination thereof, based on the total weight of the methyl glycolate oligomer.

The methyl glycolate oligomer may comprise no more than 1 wt % of a polyester based on the total weight of the methyl glycolate oligomer. The methyl glycolate oligomer may be prepared by a polycondensation reaction of a methyl glycolate composition, which may comprise no more than 1 wt % polyester based on the total weight of the composition.

The methyl glycolate oligomer may comprise no more than 1 wt % of a polyol based on the total weight of the methyl glycolate oligomer. The methyl glycolate oligomer may be prepared by a polycondensation reaction of a methyl glycolate composition, which may comprise no more 1 wt % polyol based on the total weight of the composition.

The methyl glycolate oligomer may comprise no more than 1 wt % of a polyacid based on the total weight of the methyl glycolate oligomer. The methyl glycolate oligomer may be prepared by a polycondensation reaction of a methyl glycolate composition, which may comprise no more 1 wt % polyacid based on the total weight of the composition.

The methyl glycolate oligomer may be pyrolyzed in the absence of a viscosity reducer.

The methyl glycolate oligomer may be pyrolyzed in the presence of a viscosity reducer.

The polyester may have an alkane of 2-10 carbons as a main chain and an ester group at each end of the main chain. The polyol may have an alkane of 2-10 carbons as a main chain and a hydroxyl group at each end of the main chain. The polyacid may have an alkane of 2-10 carbons as a main chain and a carboxyl group at each end of the main chain.

The polyester may be a binary ester, preferably dimethyl oxalate.

The polyol may be a dihydroxy alcohol, preferably ethylene glycol or butylene glycol.

The polyacid may be a binary acid, preferably oxalic acid.

The methyl glycolate oligomer may comprise a polyester or a copolyester, which may comprise a polyol or polyacid.

The methyl glycolate oligomer may be produced directly by transesterification of methyl glycolate in the presence of a catalyst until no methanol is distilled out.

The catalyst may be a rare earth metal catalyst. The rare earth metal catalyst may be a metal oxide, a rare earth metal inorganic salt or a rare earth metal complex comprising lanthanum (La), cerium (Ce), praseodymium (Pr), neodynium (Nd), promethium (Pm), samarium (Sm), strontium (Sr), europium (Eu), gadolinium (Gd), terbium (Tb), dysprosium (Dy), holmium (Ho), erbium (Er), thulium (Tm), ytterbium (Yb), lutetium (Lu), yttrium (Y), scandium (Sc), or a combination thereof.

The rare earth metal catalyst may be supported by an inorganic nanofiller selected from the group consisting of nano white carbon black, nano calcium carbonate, carbon nanotubes, nano fibers, and a combination thereof.

The rare earth metal catalyst may be present in an amount of 0.0001-5 wt %, preferably 0.0006-4 wt %, more preferably 0.001-2 wt %, based on the weight of the methyl glycolate.

The pyrolysis reaction may comprise: (a) heating the methyl glycolate oligomer and a viscosity reducer having a boiling point of more than 330° C. under a normal pressure or reduced pressure at 200-240° C. to make a solution; (b) heating the solution to a temperature of 230° C. or higher under a reduced pressure, whereby glycolide and a by-product are generated; (c) distilling the glycolide and the byproduct as a single distillate from the pyrolysis reaction system; (d) discharging the viscosity reducer as a non-volatile residue from the bottom of the pyrolysis reaction system; and (e) recovering the glycolide as the single distillate.

The process may further comprise feeding a fresh glycolic acid oligomer and a fresh viscosity reducer into the pyrolysis reaction system continuously or repeatedly in step (c), whereby the fresh glycolic acid oligomer is pyrolyzed continuously or repeatedly. The process may further comprise heating the methyl glycolate oligomer and a viscosity reducer having a boiling point exceeding 330° C.

The viscosity reducer may comprise no more than 1 wt % of a total content of a polyester, a polyol, a polybasic acid, based on the weight of the methyl glycolate oligomer.

The viscosity reducer may be present in an amount of 5-500 wt %, preferably 10-100 wt %, more preferably 15-60 wt %, based on the weight of the methyl glycolate oligomer.

The viscosity reducer may be a polyether polyol, preferably a polyethylene glycol, polypropylene glycol or polybutylene glycol, more preferably a polyethylene glycol having a weight average molecular weight between 1500 and 20,000, most preferably a polyethylene glycol having a weight average molecular weight of between 1,500 and 8,000.

The viscosity reducer may be a hydrocarbon mixture, preferably a mixture having a weight average molecular weight of less than 25,000 hydrocarbons, more preferably a hydrocarbon mixture having a weight average molecular weight of 1000-15,000, most preferably 1500-8000.

A composition is provided. The composition comprises greater than 90 wt % of methyl glycolate and no more than 1 wt % of a combination of the polyester, the polyol, and the polyacid, based on the weight of the composition. The composition may further comprise water or a monohydric alkyl alcohol (C₁-C₁₀).

### DETAILED DESCRIPTION OF THE INVENTION

The present invention relates to a process for preparing glycolide by pyrolyzing methyl glycolate. The inventors have discovered that, by controlling the contents of polyester, polyol, and polyacid in a pyrolysis reaction system, the residue generated by pyrolysis could be reduced and the glycolide yield could be increased. This reduces the generation of waste and increases the product yield. Because this process does not use a large amount of solvents having a high boiling point and which co-distill with the glycolide, the process can save a lot of energy and solvent recovery cost, and can be produced cost-effectively. This present invention also provides a methyl glycolate composition capable of reducing the pyrolysis residue, and is particularly suitable for use in the above process.

It is an objective of the present invention to provide a process for the preparation of a high purity, high yield glycolide which is capable of producing less solid residue during the glycolide production process. Another objective of the present invention is to provide a methyl glycolate composition as a raw material for preparing a methyl glycolate oligomer which enables the glycolide production process to produce less solid residue.

In order to achieve the above objectives, the inventors have found that as a methyl glycolate oligomer begins to degrade when heated above 200° C., its color begins to turn gradually yellow, and its color rapidly turns black when heated to 235° C. or higher. The boiling point of glycolide is 330° C. under normal pressure. The pyrolysis of a methyl glycolate oligomer to prepare glycolide often requires heating to above 230° C. under reduced pressure to obtain a high conversion rate. When industrial grade methyl glycolate (provided for industrial scale production) is used as a raw material to prepare glycolide, the pyrolysis yield is generally 80-87%, which not only causes waste of raw materials, but also produces more pyrolysis residues, increasing the cost of solid waste treatment in the later stage. The inventors have found out how to further increase the pyrolysis yield. It has been found that a high pyrolysis yield cannot be obtained because of impurities such as dimethyl oxalate and ethylene glycol contained in technical grade methyl glycolate. That is to say, the pyrolysis reaction of the synthesized methyl glycolate oligomer can be significantly improved after the content of the dimethyl oxalate and the ethylene glycol in the glycolide is kept low. The glycolide yield would be increased significantly and the pyrolysis residues would be reduced.

The inventors have also found that addition of polyfunctional esters, polyols, and polyacids to methyl glycolate with low impurity would reduce glycolide yield and increase pyrolysis residues. This further confirms the results of low pyrolysis yield and high solid residue of the product. This is because these polyesters, polyols, and polyacids are also involved in the polycondensation reaction of methyl glycolate to form a copolyester of methyl glycolate with these materials. When the pyrolysis reaction is carried out, these copolyesters are formed. The structural segment not only does not get pyrolyzed into a ring reaction, but also affects the pyrolysis and ring-forming reaction of the polyester segment of the nearby methyl glycolate, thereby reducing the yield of glycolide in the pyrolysis reaction, in addition to these copolyesters. The copolyester has a high boiling point and therefore tends to remain in the reaction unit, increasing the pyrolysis residue.

In addition, the inventors have found that if a viscosity reducer having a boiling point of more than 330° C. is introduced into the methyl glycolate oligomer, the viscosity of the reaction solution in the middle and late stages of the pyrolysis reaction can be lowered, and the effect of the mass transfer and heat transfer on the reaction can be enhanced due to the reduced viscosity, thereby resolving problems such as the reaction solution tends to create coke carbon, insufficient reaction, etc. Thus, introduction of viscosity reducer further improves the yield of glycolide in the pyrolysis reaction. These viscosity reducers are not distilled together with the glycolide, but can assist in discharging the pyrolysis residues from the transfer reaction system.

The transfer reaction system of the present invention may comprise methyl glycolate oligomers, and optionally a viscosity reducer. In the present invention, controlling the total content of the polyester, polyol, and polyacid in the pyrolysis reaction system at a low level can effectively increase the yield of glycolide in the pyrolysis reaction and reduce the pyrolysis residue. The use of a viscosity reducer in addition to reduce viscosity of the methyl glycolate oligomer in the pyrolysis reaction system also control the total content of the polyester, polyol, and polyacid.

The terms “glycolic acid oligomer” and “glycolate prepolymer” are used herein interchangeably and refer to the product synthesized from heating methyl glycolate in a transesterification reaction. The glycolic acid oligomer may have an intrinsic viscosity [q]=0.25 to 0.55 dl/g.

Unless stated otherwise, all molecular weight values are g/mol, and all average molecular weight values are number-average molecular weight values.

A process of preparing glycolide from a methyl glycolate oligomer is provided. The process comprises pyrolyzing a methyl glycolate oligomer in a pyrolysis reaction system. The pyrolysis reaction system comprises no more than 1 wt % of a polyester, a polyol, a polyacid or a combination thereof, based on the total weight of the methyl glycolate oligomer in the apparatus.

The content of the polyester in the pyrolysis reaction system containing the methyl glycolate oligomer may be controlled to be less than or equal to 1 wt %. The methyl glycolate oligomer may comprise no more than 1 wt % of a polyester based on the total weight of the methyl glycolate oligomer. The methyl glycolate oligomer may be prepared by a polycondensation reaction of a methyl glycolate composition. The methyl glycolate composition may comprise no more than 1 wt % polyester based on the total weight of the composition.

The content of the polyol in the pyrolysis reaction system containing the methyl glycolate oligomer may be controlled to be less than or equal to 1 wt %. The methyl glycolate oligomer may comprise no more than 1 wt % of a polyol based on the total weight of the methyl glycolate oligomer. The methyl glycolate oligomer may be prepared by a polycondensation reaction of a methyl glycolate composition. The methyl glycolate composition may comprise no more 1 wt % polyol based on the total weight of the composition.

The content of the polyacid in the pyrolysis reaction system containing the methyl glycolate oligomer may be controlled to be less than or equal to 1 wt %. The methyl glycolate oligomer may comprise no more than 1 wt % of a polyacid based on the total weight of the methyl glycolate oligomer. The methyl glycolate oligomer may be prepared by a polycondensation reaction of a methyl glycolate composition. The methyl glycolate composition may comprise no more 1 wt % polyacid based on the total weight of the composition.

The total content of several different combinations of the polyester or polyol or polyacid in the pyrolysis reaction system containing the methyl glycolate oligomer may be controlled to be less than or equal to 1 wt %. The methyl glycolate oligomer may comprise no more than 1 wt % of any combination of polyester, polyol and polyacid based on the total weight of the methyl glycolate oligomer. The methyl glycolate oligomer may be prepared by a polycondensation reaction of a methyl glycolate composition. The methyl glycolate composition may comprise no more 1 wt % of any combination of polyester, polyol and polyacid based on the total weight of the composition.

To prepare the methyl glycolate oligomer, a methyl glycolate composition is provided. The composition may comprise at least 90 wt %, 92%, or 95 wt % of methyl glycolate. The composition may comprise no more than 0.1 wt %, 0.5 wt % or 1 wt % of polyester, polyol, polyacid or a combination thereof. The composition may further comprise water or a monohydric alkyl alcohol (C₁-C₁₀). The composition may further comprise an inert component that does not affect the pyrolysis.

The pyrolysis process may comprise heating a methyl glycolate oligomer and a viscosity reducer having a boiling point of more than 330° C. under a normal pressure or reduced pressure at 200-240° C. to make a solution; heating the solution to a temperature of 230° C. or higher (e.g., 280° C.) under a reduced pressure, whereby glycolide and a by-product are generated; distilling the glycolide and the byproduct as a single distillate from the pyrolysis reaction system; discharging the viscosity reducer as a non-volatile residue from the bottom of the pyrolysis reaction system; and recovering the glycolide from the single distillate. The process may further comprise feeding a fresh glycolic acid oligomer and a fresh viscosity reducer into the pyrolysis reaction system continuously or repeatedly so that the fresh glycolic acid oligomer is pyrolyzed continuously or repeatedly.

The methyl glycolate composition may be heated to carry out the pyrolysis reaction in the absence of a viscosity reducer or in the presence of a viscosity reducer, the methyl glycolate composition may be heated and the boiling point is greater than 330° C. A mixture of viscosity reducers may be used to carry out the pyrolysis reaction. One preferred embodiment of the invention is as follows:

### 1. Pyrolysis Method

It should be noted that the present invention is mainly described by way of melt pyrolysis as an example, but the examples do not limit the pyrolysis method used in the present invention, and the pyrolysis methods such as solution pyrolysis and solid phase pyrolysis are equally applicable to the present invention. According to the pyrolysis method, the pyrolysis reaction system comprises a methyl glycolate oligomer. It may further comprise a viscosity reducer.

### 2. Polyesters and Polyols and Polyacids

The total content of the polyester or polyol or polyacid in the pyrolysis reaction system containing the methyl glycolate oligomer must be controlled to be less than or equal to 1 wt %. Further, the undesirable polyester, polyhydric alcohol, and polyacid each comprise a main alkyl chain of 2-10 carbons and the main alkyl chain further comprises 2 or more carboxyl groups or a hydroxyl group or an ester group in the main chain as appropriate, and are preferably selected from the group consisting of a polyester, a polyhydric alcohol, and a polyacid satisfying the above conditions. The content of dimethyl oxalate, ethylene glycol, and butylene glycol is more likely to remain than other polyesters, polyols, polyacids, dimethyl oxalate, ethylene glycol, and butylene glycol. More specifically, the commercially available low-cost industrial grade methyl glycolate contains dimethyl oxalate, ethylene glycol, butylene glycol, and especially the content of dimethyl oxalate is generally above 3 wt %.

In the present invention, the pyrolysis reaction system is preferably free of a polyester, a polyhydric alcohol, or a polyacid. However, when the total content of the polyester, polyol, and polyacid impurities in the pyrolysis reaction system is less than or equal to 1 wt %, continuing to reduce the content of these impurities cannot significantly increase the yield of glycolide and reduce the pyrolysis residue, so it is preferred that the total content of ester, polyol, and polyacid in the pyrolysis reaction system is controlled to be less than or equal to 1 wt %.

By controlling the content of polyesters, polyols, polyacids in methyl glycolate oligomers, and in other components such as viscosity reducers, it is possible to control the polyesters, polyols, and polyacids introduced to the pyrolysis reaction system. It is preferred to jointly control the total content of the polyester, the polyol, and the polyacid in the pyrolysis reaction system by using these means.

In most cases, the polyester, polyol, and polyacid are introduced into the methyl glycolate oligomer from methyl glycolate, and then introduced into the pyrolysis reaction system via the methyl glycolate oligomer. In order to control the total content of the polyester, polyol and polyacid in the pyrolysis reaction system, it is advantageous to purify the methyl glycolate, thereby reducing the total content of the polyester, the polyol and the polyacid in the methyl glycolate, and controlling at the same time the total content of these impurities to be less than or equal to 1 wt %. The purification does not lead to a significant increase in the cost of raw materials, but the production process can be controlled due to less solid residue formation and no use of high-boiling solvents, and the production of glycolide product has high purity and good yield, thereby justifying the purification step if it is done.

### 3. Methyl Glycolate Oligomers

The methyl glycolate oligomer can be prepared by a condensation reaction of glycolic acid ester, glycolic acid, or glycolate. Glycolate is preferred as the starting material, and methyl glycolate is most preferred. Because the by-product of the condensation reaction of methyl glycolate is methanol, the lower boiling point of methanol is more favorable for the removal of small molecules in the condensation reaction process to accelerate the reaction rate. The removed methanol is easy to be purified and has economic value, which can be further reduced by co-production of methanol. The production cost, the low corrosiveness of methyl glycolate, contribute to low capital requirements for equipment.

High purity methyl glycolate can be obtained by rectification, which is very suitable for industrialization.

When a component such as a viscosity reducing agent is added to the pyrolysis reaction system, the total content of the polyester, polyol, and polyacid contained in the viscosity reducing agent should be controlled to be 1 wt % or less.

The synthesis of the methyl glycolate oligomer can be carried out by first heating the methyl glycolate from 150° C. to 230° C. in 2 h, during which the pressure is reduced from high pressure to reduced pressure to below 1 kPa for 0.5 h to 2 h. The transesterification reaction is carried out until almost no methanol is distilled out of the methyl glycolate oligomer, and the addition of the transesterification catalyst can accelerate the reaction and shorten the reaction time. The obtained methyl glycolate oligomer can be directly used in the pyrolysis reaction system, and its intrinsic viscosity [η]=0.25 to 0.55 dl/9.

### 4. Catalyst for Synthesis of Methyl Glycolate Oligomer

The methyl glycolate oligomer may be prepared directly by transesterification of methyl glycolate in the presence of a catalyst until no methanol is distilled out.

The catalyst for the methyl glycolate oligomer reaction process is a rare earth metal oxide or a rare earth metal inorganic salt or a rare earth metal complex. The rare earth metal catalyst may comprise lanthanum (La), cerium (Ce), praseodymium (Pr), neodynium (Nd), promethium (Pm), samarium (Sm), strontium (Sr), europium (Eu), gadolinium (Gd), terbium (Tb), dysprosium (Dy), holmium (Ho), erbium (Er), thulium (Tm), ytterbium (Yb), lutetium (Lu), yttrium (Y), scandium (Sc), or a combination thereof.

The addition of the catalyst can efficiently prepare a methyl glycolate oligomer for subsequent pyrolysis reaction.

If necessary, in order to recover the catalyst and improve the catalytic efficiency, the rare earth metal catalyst may be supported on the inorganic nanofiller to increase its activity. The nanoparticles include: nano white carbon black, nano calcium carbonate, carbon nanotubes, and nano fibers. The rare earth metal catalyst may be added in an amount of 0.0001-5 wt %, preferably 0.0006-4 wt %, more preferably 0.001-2 wt %, based on the weight of the methyl glycolate.

### 5. Viscosity Reducer

The viscosity reducer may be a polyether polyol, preferably a polyethylene glycol, polypropylene glycol, polybutylene glycol, more preferably a polyethylene glycol having a weight average molecular weight between 1500 and 20,000, most preferably a polyethylene glycol having a weight average molecular weight of between 1,500 and 8,000. The polyethylene glycol may be added in an amount of about 5-500 wt %, preferably 10-100 wt %, more preferably 15-60 wt %, based on the weight of the methyl glycolate oligomer.

The viscosity reducer may be a hydrocarbon mixture, preferably a mixture having a weight average molecular weight of less than 25,000 hydrocarbons, more preferably a hydrocarbon mixture having a weight average molecular weight of 1000-15,000, most preferably 1500-8000. The hydrocarbon mixture may be added in an amount of about 5-500 wt %, preferably 10-100 wt %, more preferably 15-60 wt %, based on the weight of the methyl glycolate oligomer.

These viscosity reducers may be heated at 250° C. under an absolute pressure of 1 kPa for 1 hour. It has been found that the viscosity reducers minimally degraded, if at all and their efficacy remained high. The viscosity reducer therefore can be used repeatedly.

### 6. Conditions for Pyrolysis

In the pyrolysis reaction system, heating may be carried out under a reduced pressure, preferably under a reduced pressure of about 0.1-20 kPa absolute pressure, more preferably 0.1-10 kPa, most preferably 0.1-3 kPa. The heating temperature may be at least 230° C., preferably 230-270° C., more preferably 230-265° C. A high temperature above 270° C. is tends to increase carbonization of the polymer, which is not conducive to the pyrolysis reaction.

The viscosity reducer in the pyrolysis system is not co-distilled with glycolide. The distilled glycolide may be recrystallized or distilled to obtain high quality purified glycolide, for example, a purity greater than 90 wt %, 91 wt %, 92 wt %, 93 wt %, 94 wt %, 95 wt %, 96 wt %, 97 wt %, 98 wt %, 99 wt %, 99.5 wt % or 99.9 wt %; and a free acid content no more than 150 ppm, 200, 250 or 300 ppm, based on the weight of the methyl glycolate oligomer. Compared with the traditional reaction system in which the solvent and the glycolide are co-distilled, this process not requires solvent evaporation, separation of solvent from glycolide, and solvent recovery.

### 7. Methyl Glycolate Composition

In order to synthesize a methyl glycolate oligomer satisfying the requirements of a pyrolysis reaction system, the methyl glycolate composition used in the present invention may contain at least 90 wt %, 92%, or 95 wt % of methyl glycolate. The composition may comprise no more than 0.1 wt %, 0.5 wt % or 1 wt % of polyester, polyol, polyacid or a combination thereof. The remaining components of the composition may be water or a monohydric alkyl alcohol (C₁-C₁₀) or an inert component which does not participate in the reaction.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Glycolide Preparation

The present invention will be specifically explained below in conjunction with specific embodiments.

### A. Test Methods

1. Determination of Free Acid Content in Glycolide

A 0.5 g sample was weighed into a conical flask, about 20 ml of dimethyl sulfoxide was added, and the glycolide solution was subjected to potentiometric titration with a solution of 0.01 mol/L potassium hydroxide.

2. Determination of Inherent Viscosity of Methyl Glycolate Oligomer

A sample of about 0.125 g was weighed, dissolved in 25 ml of hexafluoroisopropanol, and subjected to a constant temperature water bath at 25° C. The inherent viscosity (q) was measured using an Ubbelohde viscometer. The average was measured three times. The outflow time of each measurement did not differ by more than 0.2 s.

### B. Methyl Glycolate Compositions

21 methyl glycolate (MG) compositions were prepared and tested. The test results are shown in Table 1.

MG 1: 1000 g methyl glycolate (MG) was placed in a 5 L autoclave. The temperature of the autoclave was gradually increased from 150° C. to 230° C. within 2 h while the pressure was gradually reduced from 450 kPa to 1 kPa during this time period. The residence time below 1 kPa was 0.5 h. The resulting methyl glycolate oligomer was marked as a, and measured for its inherent viscosity.

MG 2: Methyl glycolate oligomer b was prepared using the same preparation method for methyl glycolate oligomer a except that residence time was 1 h.

MG 3: Methyl glycolate oligomer c was prepared using the same preparation method for methyl glycolate oligomer a except that residence time was 1.5 h.

MG 4: Methyl glycolate oligomer d was prepared using the same preparation method for methyl glycolate oligomer a except that residence time was 2 h.

MG 5: Methyl glycolate oligomer e was prepared using the same preparation method for methyl glycolate oligomer b except that 990 g methyl glycolate and 10 g dimethyl oxalate were placed in the autoclave.

MG 6: Methyl glycolate oligomer f was prepared using the same preparation method for methyl glycolate oligomer b except that 990 g methyl glycolate and 10 g ethylene glycol were placed in the autoclave.

MG 7: Methyl glycolate oligomer g was prepared using the same preparation method for methyl glycolate oligomer b except that 990 g methyl glycolate and 10 g oxalic acid were placed in the autoclave.

MG 8: Methyl glycolate oligomer h was prepared using the same preparation method for methyl glycolate oligomer b except that 990 g methyl glycolate, 5 g oxalic acid and 5 g dimethyl oxalate were placed in the autoclave.

MG 9: Methyl glycolate oligomer i was prepared using the same preparation method for methyl glycolate oligomer b except that 990 g methyl glycolate, 3 g oxalic acid, 3 g dimethyl oxalate and 4 g ethylene glycol were placed in the autoclave.

MG 10: 990 g methyl glycolate was placed in a 5 L autoclave. The temperature of the autoclave was gradually increased from 150° C. to 230° C. within 2 h while the pressure was gradually reduced from 450 kPa to 1 kPa during this time period. The residence time below 1 kPa was 1 h. 10 g dimethyl oxalate was added. Stirring was continued for 0.5 h. The resulting methyl glycolate oligomer was marked as j, and measured for its inherent viscosity.

MG 11: Methyl glycolate oligomer k was prepared using the same preparation method for methyl glycolate oligomer j except that 10 g dimethyl oxalate, instead of 10 g dimethyl oxalate, was added.

MG 12: Methyl glycolate oligomer l was prepared using the same preparation method for methyl glycolate oligomer j except that 5 g ethylene glycol and 5 g oxalic acid, instead of 10 g dimethyl oxalate, was added.

MG 13: Methyl glycolate oligomer m was prepared using the same preparation method for methyl glycolate oligomer j except that 3 g ethylene glycol, 3 g oxalic acid and 4 g dimethyl oxalate, instead of 10 g dimethyl oxalate, was added.

MG 14: Methyl glycolate oligomer n was prepared using the same preparation method for methyl glycolate oligomer b except that 990 g methyl glycolate, 3 g adipic acid, 3 g dimethyl malonate and 4 g butanediol were placed in the autoclave.

MG 15: Methyl glycolate oligomer o was prepared using the same preparation method for methyl glycolate oligomer a except that 990 g methyl glycolate, 10 g ethylene glycol, and 0.01 g La₂O₃ were placed in the autoclave.

MG 16: Methyl glycolate oligomer p was prepared using the same preparation method for methyl glycolate oligomer a except that 990 g methyl glycolate, 10 g ethylene glycol, and 0.01 g Ce(HCO₃)₄ were placed in the autoclave.

MG 17: Methyl glycolate oligomer q was prepared using the same preparation method for methyl glycolate oligomer a except that 990 g methyl glycolate, 10 g ethylene glycol, and 0.01 g×tris (cyclopentadienyl) lanthanum and [III] were placed in the autoclave.

MG 18: Methyl glycolate oligomer r was prepared using the same preparation method for methyl glycolate oligomer a except that 990 g methyl glycolate, 10 g ethylene glycol, and 0.001 g Ce(HCO₃)₄ were placed in the autoclave.

MG 19: Methyl glycolate oligomer s was prepared using the same preparation method for methyl glycolate oligomer a except that 990 g methyl glycolate, 10 g ethylene glycol, and 0.0002 g Ce(HCO₃)₄ were placed in the autoclave.

MG 20: Methyl glycolate oligomer t was prepared using the same preparation method for methyl glycolate oligomer a except that 990 g methyl glycolate, 10 g ethylene glycol, and 4 g Ce(HCO₃)₄ were placed in the autoclave.

MG 21: Methyl glycolate oligomer u was prepared using the same preparation method for methyl glycolate oligomer a except that 990 g methyl glycolate, 10 g ethylene glycol, 0.01 g Ce(HCO₃)₄, and nano-silica were placed in the autoclave.

### C. Pyrolysis

16 pyrolysis products were prepared as described below and tested. The test results are shown in Table 2.

Pyrolysis samples 1-14: 100 g methyl glycolate oligomer a-n was heated to 250° C. under stirring, and pyrolyzed for 1 h under absolute pressure of 1 kPa. The distillate was intercepted with ice water. The mass of the distillate and the quality of the pyrolysis residue were recorded. The glycolide content and the free acid content in the distillate were tested to calculate the residual amount (%) and the glycolide yield. The distillate was recrystallized in ethyl acetate to obtain purified glycolide product.

Pyrolysis sample 15: 100 g methyl glycolate oligomer l was stirred with 40 g polyethylene glycol (PEG 1500, average molecular weight 1500) (viscosity reducer) to 225° C. for 0.5 h, and then heated to 250° C. under stirring, and pyrolyzed for 1 h under absolute pressure of 1 kPa. The distillate was condensed with ice water. The mass of the distillate and the quality of the pyrolysis residue (excluding viscosity reducer) were recorded. The glycolide content and the free acid content in the distillate were tested to calculate the residual amount (%) and the glycolide yield. The distillate was recrystallized in ethyl acetate to obtain purified glycolide product.

Pyrolysis sample 16: 100 g methyl glycolate oligomer l was stirred with 40 g paraffin wax (average molecular weight 1000) (viscosity reducer) to 225° C. for 0.5 h, and then heated to 250° C. under stirring, and pyrolyzed for 1 h under absolute pressure of 1 kPa. The distillate was condensed with ice water. The mass of the distillate and the quality of the pyrolysis residue (excluding viscosity reducer) were recorded. The glycolide content and the free acid content in the distillate were tested to calculate the residual amount (%) and the glycolide yield. The distillate was recrystallized in ethyl acetate to obtain purified glycolide product.

### D. Comparative Samples

Comparative samples 1 and 2 were prepared as described below and tested. The test results are shown in Tables 1 and 2.

Comparative sample 1 (C1): 985 g methyl glycolate, 5 g oxalic acid, 5 g dimethyl oxalate and 5 g ethylene glycol were placed in a 5 L autoclave. The temperature of the autoclave was gradually increased from 150° C. to 230° C. within 2 h while the pressure was gradually reduced from 450 kPa to 1 kPa during this time period. The residence time below 1 kPa was 1 h. The resulting methyl glycolate oligomer was marked as v, and measured for its inherent viscosity.

100 g methyl glycolate oligomer v was heated to 250° C. under stirring, and pyrolyzed for 1 h under absolute pressure of 1 kPa. The distillate was condensed with ice water. The mass of the distillate and the quality of the pyrolysis residue were recorded. The glycolide content and the free acid content in the distillate were tested to calculate the residual amount (%) and the glycolide yield. The distillate was recrystallized in ethyl acetate to obtain purified glycolide product

Comparative sample 2 (C2): 970 g methyl glycolate, 5 g oxalic acid, 20 g dimethyl oxalate and 5 g ethylene glycol were placed in a 5 L autoclave. The temperature of the autoclave was gradually increased from 150° C. to 230° C. within 2 h while the pressure was gradually reduced from 450 kPa to 1 kPa during this time period. The residence time below 1 kPa was 1 h. The resulting methyl glycolate oligomer was marked as w, and measured for its inherent viscosity.

100 g methyl glycolate oligomer w was heated to 250° C. under stirring, and pyrolyzed for 1 h under absolute pressure of 1 kPa. The distillate was condensed with ice water. The mass of the distillate and the quality of the pyrolysis residue were recorded. The glycolide content and the free acid content in the distillate were tested to calculate the residual amount (%) and the glycolide yield. The distillate was recrystallized in ethyl acetate to obtain purified glycolide product

TABLE 1
Methyl glycolate compositions

| No. | MG composition | MG Oligomer type | Inherent Viscosity (dL/g) |
|---|---|---|---|
| 1 | MG1 | a | 0.26 |
| 2 | MG2 | b | 0.33 |
| 3 | MG3 | c | 0.42 |
| 4 | MG4 | d | 0.52 |
| 5 | MG5 | e | 0.31 |
| 6 | MG6 | f | 0.32 |
| 7 | MG7 | g | 0.33 |
| 8 | MG8 | h | 0.32 |
| 9 | MG9 | i | 0.32 |
| 10 | MG10 | j | 0.31 |
| 11 | MG11 | k | 0.33 |
| 12 | MG12 | l | 0.33 |
| 13 | MG13 | m | 0.32 |
| 14 | MG14 | n | 0.31 |
| 15 | MG15 | o | 0.48 |
| 16 | MG16 | p | 0.53 |
| 17 | MG17 | q | 0.50 |
| 18 | MG18 | r | 0.51 |
| 19 | MG19 | s | 0.46 |
| 20 | MG20 | t | 0.54 |
| 21 | MG21 | u | 0.58 |
| C1 | MG21 | v | 0.32 |
| C2 | MG22 | w | 0.31 |

TABLE 2
Pyrolysis Samples

| | | | | | | | | Free Acid |
|---|---|---|---|---|---|---|---|---|
| | MG oligomer | Polyester | Polyol | Polyacid | Viscosity | Remaining | Glycolide | Content |
| No. | type | (%) | (%) | (%) | Reducer | Residue | Yield | (ppm) |
| 1 | a | — | — | — | — | 1.21% | 95.74% | 2453 |
| 2 | b | — | — | — | — | 1.63% | 97.65% | 1898 |
| 3 | c | — | — | — | — | 2.12% | 97.29% | 2001 |
| 4 | d | — | — | — | — | 2.54% | 97.01% | 2337 |
| 5 | e | 1 | — | — | — | 3.93% | 94.55% | 2437 |
| 6 | f | — | 1 | — | — | 3.88% | 95.45% | 2544 |
| 7 | g | — | — | 1 | — | 3.99% | 94.32% | 2777 |
| 8 | h | 0.5 | — | 0.5 | — | 3.94% | 94.22% | 2543 |
| 9 | i | 0.3 | 0.4 | 0.3 | — | 3.94% | 95.35% | 2646 |
| 10 | j | 1 | — | — | — | 3.89% | 94.98% | 2348 |
| 11 | k | — | 1 | — | — | 3.78% | 95.33% | 2701 |
| 12 | l | — | 0.5 | 0.5 | — | 3.93% | 94.47% | 2656 |
| 13 | m | 0.4 | 0.3 | 0.3 | — | 3.78% | 95.08% | 2787 |
| 14 | n | 0.3 | 0.4 | 0.3 | — | 3.86% | 95.16% | 2699 |
| 15 | l | — | 0.5 | 0.5 | 40% | 1.89% | 97.79% | 2031 |
| | | | | | PEG100 | | | |
| | | | | | 0 | | | |
| 16 | l | — | 0.5 | 0.5 | 40% | 2.01% | 97.54% | 1999 |
| | | | | | paraffin | | | |
| C1 | o | 0.5 | 0.5 | 0.5 | — | 7.47% | 91.21% | 2778 |
| C2 | p | 2 | 0.5 | 0.5 | — | 10.83% | 88.33% | 2987 |

As shown in above, the content of the polyol, the polyacid or the polyester in the reaction system has a direct influence on the yield of the glycolide product, the amount of the remaining solid residue, and the purity of the glycolide. When the total amount of these three impurities was less than 1%, there appears to be no obvious effect on the yield. Moreover, the yield of the glycolide product, the amount of the remaining solid residue, and the purity of the glycolide closely correlated to the preparation method of the glycolide. The addition of a viscosity reducer was more advantageous for the progress of the pyrolysis reaction, reducing the remaining solid residue and increasing the glycolide yield.

Although the invention is illustrated and described herein with reference to specific embodiments, the invention is not intended to be limited to the details shown. Rather, various modifications may be made in the details within the scope and range of equivalents of the claims without departing from the invention.

1. A process of preparing glycolide from a methyl glycolate oligomer, comprising pyrolyzing a methyl glycolate oligomer in a pyrolysis reaction system, wherein the pyrolysis reaction system comprises no more than 1 wt % of a polyester, a polyol, a polyacid or a combination thereof, based on the total weight of the methyl glycolate oligomer.

2. The process of claim 1, wherein the methyl glycolate oligomer comprises no more than 1 wt % of a polyester based on the total weight of the methyl glycolate oligomer.

3. The process of claim 2, wherein the methyl glycolate oligomer is prepared by a polycondensation reaction of a methyl glycolate composition comprising no more than 1 wt % polyester based on the total weight of the composition.

4. The process of claim 1, wherein the methyl glycolate oligomer comprises no more than 1 wt % of a polyol based on the total weight of the methyl glycolate oligomer.

5. The process of claim 4, wherein the methyl glycolate oligomer is prepared by a polycondensation reaction of a methyl glycolate composition comprising no more 1 wt % polyol based on the total weight of the composition.

6. The process of claim 1, wherein the methyl glycolate oligomer comprises no more than 1 wt % of a polyacid based on the total weight of the methyl glycolate oligomer.

7. The process of claim 6, wherein the methyl glycolate oligomer is prepared by a polycondensation reaction of a methyl glycolate composition comprising no more 1 wt % polyacid based on the total weight of the composition.

8. The process of claim 1, wherein the methyl glycolate oligomer is pyrolyzed in the absence of a viscosity reducer.

9. The process of claim 1, wherein the methyl glycolate oligomer is pyrolyzed in the presence of a viscosity reducer.

10. The process of claim 1, wherein the polyester has an alkane of 2-10 carbons as a main chain and an ester group at each end of the main chain, wherein the polyol has an alkane of 2-10 carbons as a main chain and a hydroxyl group at each end of the main chain, and wherein the polyacid has an alkane of 2-10 carbons as a main chain and a carboxyl group at each end of the main chain.

11. The process of claim 1, wherein the polyester is a binary ester.

12. The process of claim 1, wherein the polyol is a dihydroxy alcohol.

13. The process of claim 1, wherein the polyacid is a binary acid.

14. The process of claim 1, wherein the methyl glycolate oligomer comprises a polyester or a copolyester comprising a polyol or polyacid.

15. The process of claim 1, wherein the methyl glycolate oligomer is produced directly by transesterification of methyl glycolate in the presence of a catalyst until no methanol is distilled out of the methyl glycolate oligomer.

16. The process of claim 15, wherein the glycolic acid oligomer has an intrinsic viscosity [η]=0.25 to 0.55 dl/g.

17. The process of claim 15, wherein the catalyst is a rare earth metal catalyst, wherein the rare earth metal catalyst is a metal oxide, a rare earth metal inorganic salt or a rare earth metal complex comprising lanthanum (La), cerium (Ce), praseodymium (Pr), neodynium (Nd), promethium (Pm), samarium (Sm), strontium (Sr), europium (Eu), gadolinium (Gd), terbium (Tb), dysprosium (Dy), holmium (Ho), erbium (Er), thulium (Tm), ytterbium (Yb), lutetium (Lu), yttrium (Y), scandium (Sc), or a combination thereof.

18. The process of claim 17, wherein the rare earth metal catalyst is supported by an inorganic nanofiller selected from the group consisting of nano white carbon black, nano calcium carbonate, carbon nanotubes, nano fibers, and a combination thereof.

19. The process of claim 17, wherein the rare earth metal catalyst is present in an amount of 0.0001-5 wt % based on the weight of the methyl glycolate.

20. The process of claim 1, wherein the pyrolysis comprises:
(a) heating the methyl glycolate oligomer and a viscosity reducer having a boiling point of more than 330° C. under a normal pressure or reduced pressure at 200-240° C. to make a solution;
(b) heating the solution to a temperature of 230° C. or higher under a reduced pressure, whereby glycolide and a by-product are generated;
(c) distilling the glycolide and the by-product as a single distillate from the pyrolysis reaction system;
(d) discharging the viscosity reducer as a non-volatile residue from the bottom of the pyrolysis reaction system; and
(e) recovering the glycolide from the single distillate.

21. The process of claim 20, further comprising feeding a fresh glycolic acid oligomer and a fresh viscosity reducer into the pyrolysis reaction system continuously or repeatedly in step (c), whereby the fresh glycolic acid oligomer is pyrolyzed continuously or repeatedly.

22. The process of claim 1, further comprising heating the methyl glycolate oligomer and a viscosity reducer having a boiling point exceeding 330° C.

23. The process of claim 9, wherein the viscosity reducer comprises no more than 1 wt % of a total content of a polyester, a polyol, a polybasic acid, based on the weight of the methyl glycolate oligomer.

24. The process of claim 9, wherein the viscosity reducer is present in an amount of 5-500 wt % based on the weight of the methyl glycolate oligomer.

25. The process of claim 9, wherein the viscosity reducer is a polyether polyol.

26. The process of claim 9, wherein the viscosity reducer is a hydrocarbon mixture.

27. A composition comprising greater than 90 wt % of methyl glycolate and no more than 1 wt % of a combination of the polyester, the polyol, and the polyacid based on the weight of the methyl glycolate oligomer.

28. The composition of claim 27, further comprising water or a monohydric alkyl alcohol (C₁-C₁₀).
